(19) United States
(12) Patent Application Publication
(10) Pub. No.: US 2022/0296222 A1
(43) Pub. Date: Sep. 22, 2022

(54) Dual Syringe for Use with Percutaneous Aspirating Cannula

(71) Applicant: Adimend Biosciences, LLC, San Antonio, TX (US)

(72) Inventor: Pendleton WICKERSHAM, San Antonio, TX (US)

(21) Appl. No.: 17/687,798

(22) Filed: Mar. 7, 2022

Related U.S. Application Data
(63) Continuation-in-part of application No. 16/448,758, filed on Jun. 21, 2019.
Application No. 16/448,758 is a continuation of application No. 15/606,641, filed on May 26, 2017, now Pat. No. 10,349,924.
Application No. 15/606,641 is a continuation of application No. 15/502,814, filed on Feb. 9, 2017, now abandoned.
(60) Provisional application No. 62/092,022, filed on Dec. 15, 2014.
(60) Provisional application No. 62/022,511, filed on Jul. 9, 2014.

(51) Int. Cl. A61B 10/02; A61M 5/32; A61M 5/19; A61M 5/31

(52) U.S. Cl. CPC A61B 10/0283; A61M 5/3293; A61M 5/19; A61B 10/0275; A61M 5/3134

(57) ABSTRACT

The dual syringe with percutaneous cannula is a medical device for harvesting and transferring cells for autologous transplantation without the requirement of a surgical setting or ambulatory care. The dual syringe is a closed sterile system that allows for the harvesting syringe to harvest the cells/tissue from a first location of a patient which is then centrifuged to aggregate the cells. The aggregated cell pellet is transferred to a transfer syringe. The cell pellet is then reinjected by the transfer syringe into the patient at a second location for therapeutic purpose. The percutaneous cannula permits insertion of a cannula, with the assistance of a needle, without the need of a surgical incision or trocar.

### CROSS REFERENCE TO RELATED APPLICATIONS

This application claims the benefit of the following: U.S. patent application Ser. No. 16/448,758 filed Jun. 21, 2019; U.S. patent application Ser. No. 15/721,088 filed Sep. 29, 2017; U.S. patent application Ser. No. 15/721,155 filed Sep. 29, 2017; U.S. patent application Ser. No. 15/606,641 filed May 26, 2017; U.S. patent application Ser. No. 15/502,814 filed Feb. 9, 2017; PCT Application Serial No. PCT/US2015/039833 filed Jul. 9, 2015; U.S. Provisional Application Ser. No. 62/092,022 filed Dec. 15, 2014; and U.S. Provisional Application Ser. No. 62/022,511 filed Jul. 9, 2014; which are all incorporated by reference herein.

### BACKGROUND OF THE INVENTION

### 1. Field of the Invention

The present invention relates to the field of medical devices, specifically syringes and aspirating cannulas.

### 2. Description of the Related Art

Autologous stem cell transplant generally occurs in a highly sterile environment such as available in an operating room or ambulatory surgical center. One of the common methods to obtain stem cells is through the use of aspirating cannulas. However, cannulas require a second instrument to access the interior of the body for collection of the stem cells. For example, extraction of bone marrow stem cells generally requires the insertion of a trocar and the cannula after the trocar is in place. Similarly, extraction of stem cells from adipose tissues generally requires a scalpel incision into the patient before insertion of the aspirating cannula. Moreover, once the desired cells and tissues are harvested by the cannula, the cells or tissues are then transferred to syringes or test tubes. These transfers significantly increase the opportunity for sterile breaks through introduction of manual transfers. An invention and method is desired to simply extract and collect desired cells from a patient in a sterile closed system and then reintroduce the concentrated cells for therapeutic purpose.

The present invention simplifies the process for autologous stem cell transplant by utilization of a dual syringe with percutaneous aspirating cannula that safely harvests cells and tissues without the need for a surgical procedure and by minimizing the opportunity for a sterile break. The percutaneous aspirating cannula utilizes a needle and a tapered edge cannula wherein the needle and tapered edge allow for the percutaneous aspirating cannula to be inserted directly into a patient without the need of surgical incision or insertion of a trocar. The dual syringe is a closed system that allows for the harvesting syringe to harvest the cells/tissue which is then centrifuged to aggregate the cells wherein the isolated cell pellet is then transferred to a second syringe where it can then be reinjected to the patient for a desired therapeutic purpose.

### BRIEF SUMMARY OF THE DRAWINGS

FIG. 1A is a perspective view of a first preferred embodiment of the dual syringe assembly of the present invention.

FIG. 1B is a perspective view of the first preferred embodiment of the dual syringe assembly of the present invention as shown in FIG. 1A with the end cover removed.

FIG. 1C is an exploded perspective view of the first preferred embodiment of the dual syringe assembly of the present invention as shown in FIG. 1A.

FIG. 2A is a perspective view of the first preferred embodiment of the dual syringe assembly of the present invention as shown in FIG. 1A with the end cover, luer stop, valve components, and gutter cap removed for clarity.

FIG. 2B is a perspective view of the interior of the gutter cap of the first preferred embodiment of the dual syringe assembly of the present invention.

FIG. 2C is an end view of the first preferred embodiment of the dual syringe assembly of the present invention as shown in FIG. 1A with the end cover, luer stop, valve components, and gutter cap removed for clarity.

FIGS. 3A-3D are detailed perspective views of the syringe components of the first preferred embodiment of the dual syringe assembly of the present invention as shown in FIG. 1A.

FIG. 4A is a cross sectional view of the first preferred embodiment of the dual syringe assembly of the present invention as shown in FIG. 1A.

FIG. 4B is a detailed cross sectional view of the small syringe-riser tube connection of the first preferred embodiment of the dual syringe assembly of the present invention.

FIG. 4C is a detailed cross sectional view of end portion of the first preferred embodiment of the dual syringe assembly of the present invention.

FIG. 5A is a cross section view of the device of the present invention configured to begin harvesting with the attachable cannula removed for clarity.

FIG. 5B is a cross section view of the device of the present invention configured post harvesting with the large syringe drawn out (once again the attachable cannula is removed for clarity).

FIG. 5C is a cross section view of the device of the present invention configured post harvesting with the large syringe drawn out and the luer stop positioned in place of the attachable cannula, the device prepared for centrifuging.

FIG. 5D is a cross section view of the device of the present invention configured post centrifuging with the large syringe pushed partially inward, opening the check valve and initiating a flow into the gutter chambers.

FIG. 5E is a cross section view of the device of the present invention configured post centrifuging with the large syringe pushed partially inward, with the transfer flow moving through the riser tube into the small syringe.

FIG. 5F is a cross section view of the device of the present invention configured post transfer with the large syringe pushed fully inward, the transfer flow moving through the riser tube to fill the small syringe.

FIG. 5G is a cross section view of the device of the present invention configured for centrifuging with the large syringe shaft removed before centrifuging begins.

FIG. 5H is a cross section view of the device of the present invention configured immediately post centrifuging with the large syringe shaft removed and the cell pellet material forced to the lower part of the large syringe.

FIG. 6 is a cross sectional view of a second preferred embodiment of the dual syringe assembly of the present invention with a lower step riser tube and an elongated transfer syringe.

FIG. 7 is a profile view of the needle hub assembly of the present invention.

FIG. 8 is a profile view of the cannula component of the present invention.

FIG. 9 is a profile view of the assembly of the needle hub and percutaneous cannula of the present invention.

FIG. 10 is a detailed cross section of the side profile of the needle hub assembly of the present invention.

FIG. 11 is a flow chart diagram showing the steps in the method of autologous stem cell transplant of the present invention.

### DETAILED DESCRIPTION OF THE VARIOUS EMBODIMENTS

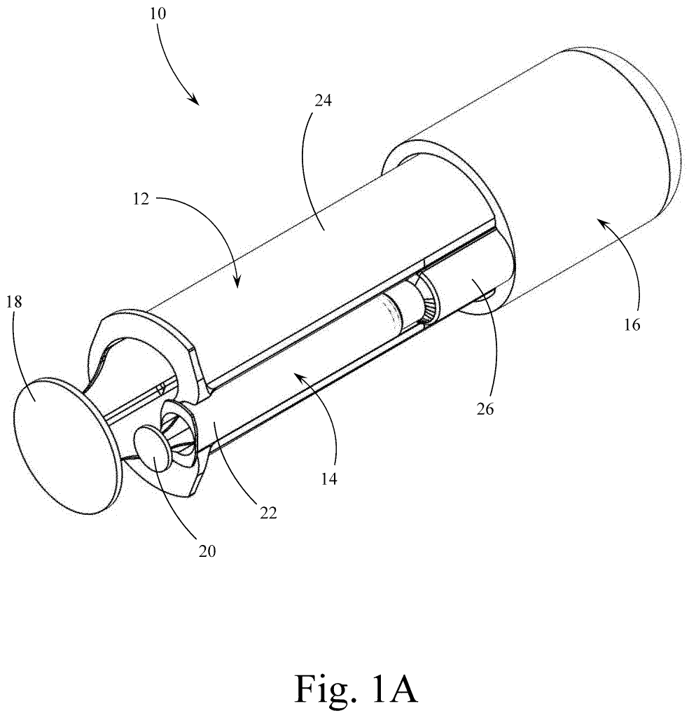

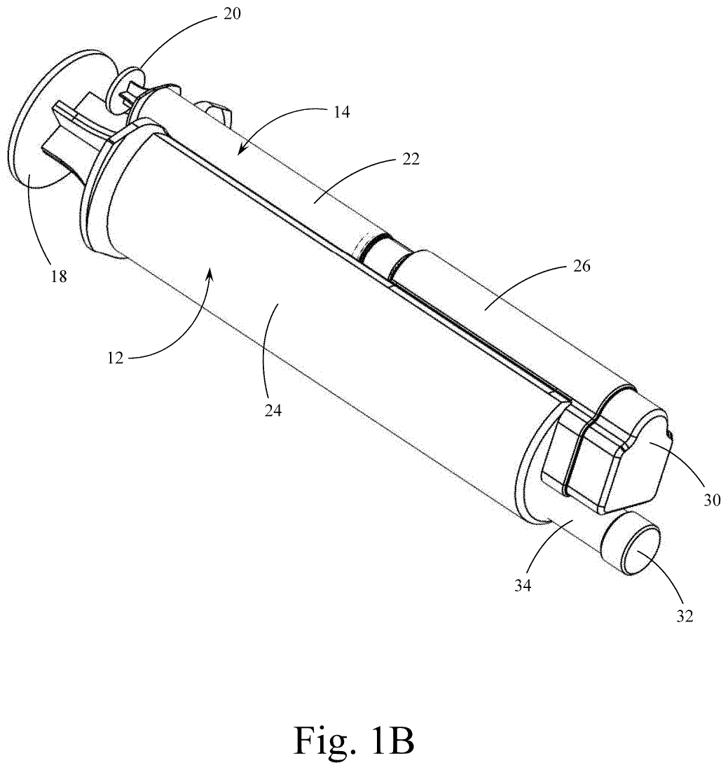

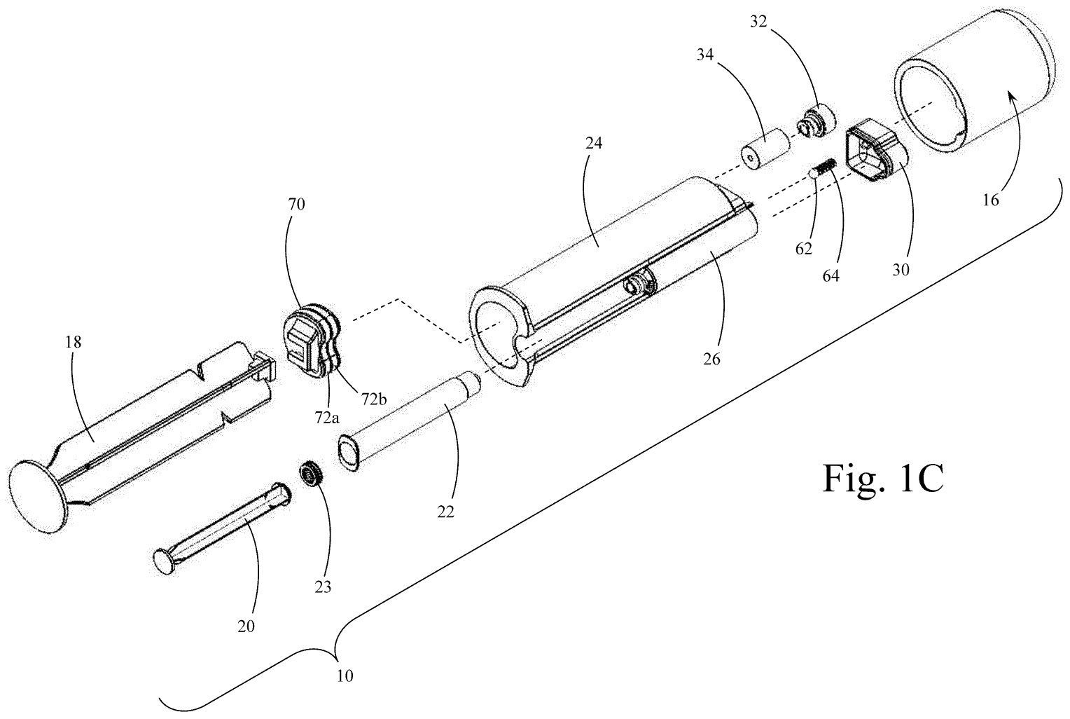

Reference is made first to FIG. 1A which is a perspective view of a first preferred embodiment of the dual syringe assembly of the present invention. Dual syringe assembly 10 includes large (harvesting) syringe 12 (60 cc typical) and small (transfer) syringe 14 (3 cc typical). In its compact assembly prior to use the dual syringe is covered with end cover (sabot) 16. Large (harvesting) syringe 12 is generally made up of large (harvesting) syringe shaft 18 and large (harvesting) syringe tube 24. Small (transfer) syringe 14 is generally made up of small (transfer) syringe shaft 20 and small (transfer) syringe tube 22. Small syringe 14 is removably positioned in a hemicylindrical channel formed along the length of large syringe 12 (see FIG. 1C). The difference in overall length between large syringe 12 and small syringe 14 is made up by riser tube 26 which extends up from a common base (covered with end cover 16 in FIG. 1A) to a point of attachment for small syringe 14. This arrangement permits large syringe shaft 18 and small syringe shaft 20 to be positioned proximate to each other for handling, use and operation of the dual syringe.

FIG. 1B is a perspective view of the first preferred embodiment of the dual syringe assembly of the present invention as shown in FIG. 1A with the end cover removed. Once again, the dual syringe is shown to include large (harvesting) syringe 12 and small (transfer) syringe 14. In the view shown in FIG. 1B, the end cover (sabot) 16 is removed to show the lower components of the dual syringe as immediately prior to use. Once again, large syringe 12 is the combination of large syringe shaft 18 inserted fully (in this view prior to use) into large syringe tube 24. Once again, small syringe 14 is the combination of small syringe shaft 20 inserted fully (in this view prior to use) into small syringe tube 22.

The lower end of large syringe tube 24 transitions into luer (cannula connector) 34 which, in the view of FIG. 1B, is covered with luer stop (cannula connector cover) 32. The lower end of small syringe tube 22 connects to riser tube 26 which terminates inside gutter cap 30 in a manner described in more detail below.

FIG. 1C is an exploded perspective view of the first preferred embodiment of the dual syringe assembly of the present invention as shown in FIG. 1A. Dual syringe assembly 10 generally comprises a linear assembly of components extending from large (harvesting) syringe shaft 18 and small (transfer) syringe shaft 20 through to end cover (sabot) 16. Large syringe shaft 18 is removably attached to large syringe plunger 70 which holds large syringe plunger gasket rings 72a & 72b. The separable attachment components are preferably a slotted arrangement that allows the shaft to be slid to the slide (when the shaft is sufficiently drawn out from the syringe tube) and disconnected from the plunger. The shape of the large syringe plunger 70 accommodates the interior cross section of large syringe tube 24 into which the shaft and plunger are inserted. This rounded crescent shape, as shown in FIG. 1C, not only accommodates the tube side channel that holds the small (transfer) syringe but also accommodates the transfer structures interior at the base of the dual syringe. Small syringe shaft 20 is attached to small syringe plunger 23 in a manner typical of most cylindrical syringes. The combination of shaft 20 and plunger 23 are inserted into small syringe tube 22. Small syringe tube 22 is attached to riser tube 26 (integral or attached to large syringe tube 24) by a threaded connection or other twist to lock syringe connectors.

Positioned on the base of the large syringe tube is luer (cannula connector) 34 which, at various steps in the process during use, is covered and sealed with luer stop (cannula connector cover) 32. Also positioned on the base of the large syringe tube, in a manner described in more detail below, are transfer valve ball 62 and spring 64. Covering the ball valve assembly and providing a configured internal flow channel is gutter cap 30. The manner in which collected and centrifuged tissue cells move from the large harvesting syringe to the small transfer syringe by way of the chamber and channels defined by gutter cap 30 is described in more detail below.

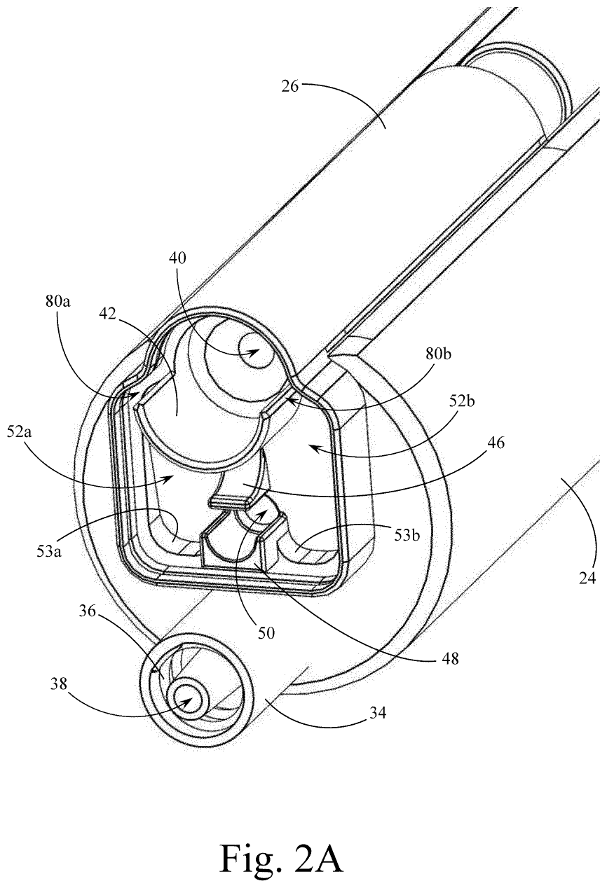

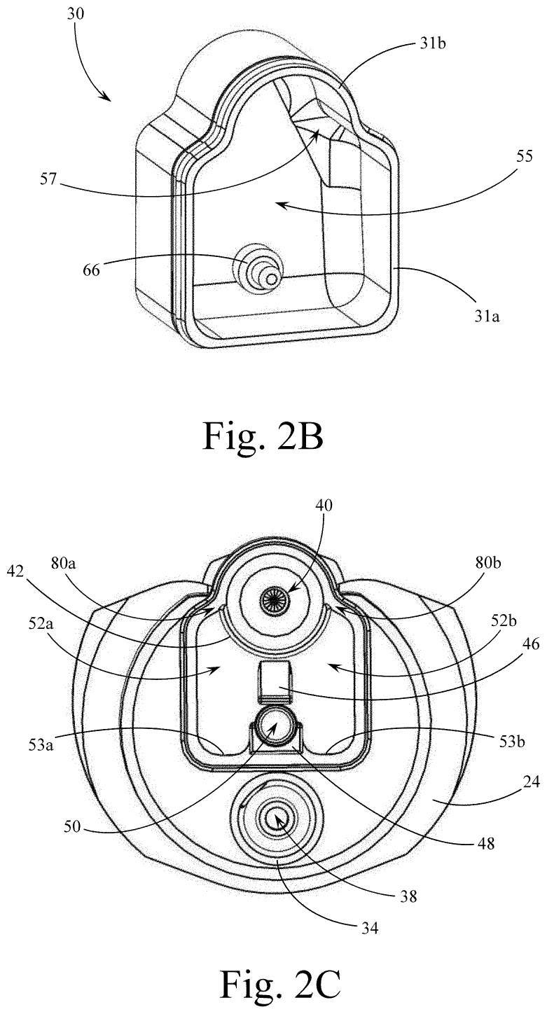

FIG. 2A is a perspective view of the first preferred embodiment of the dual syringe assembly of the present invention as shown in FIG. 1A with the end cover, luer stop, valve components, and gutter cap removed for clarity. In this view, the lower ends of large (harvesting) syringe tube 24 and riser tube 26 are shown. As described above, large syringe tube 24 transitions into luer (cannula connector) 34 which is internally configured with luer port 38 and cannula attachment threads 36.

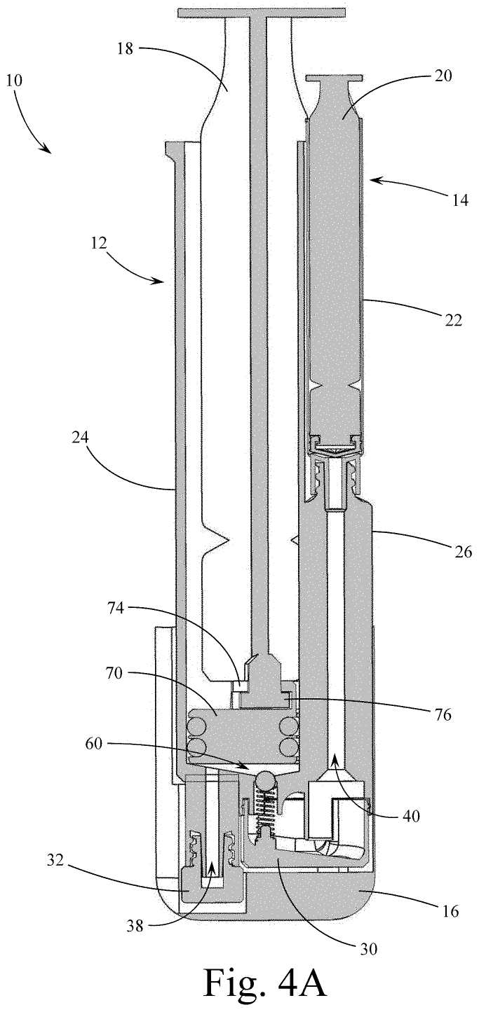

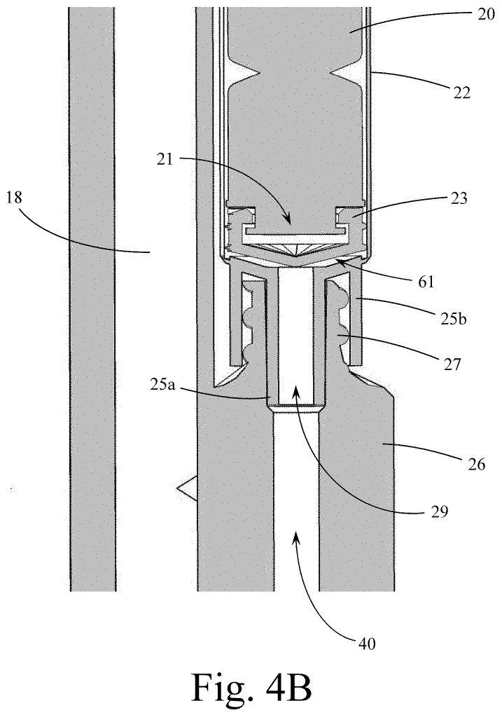

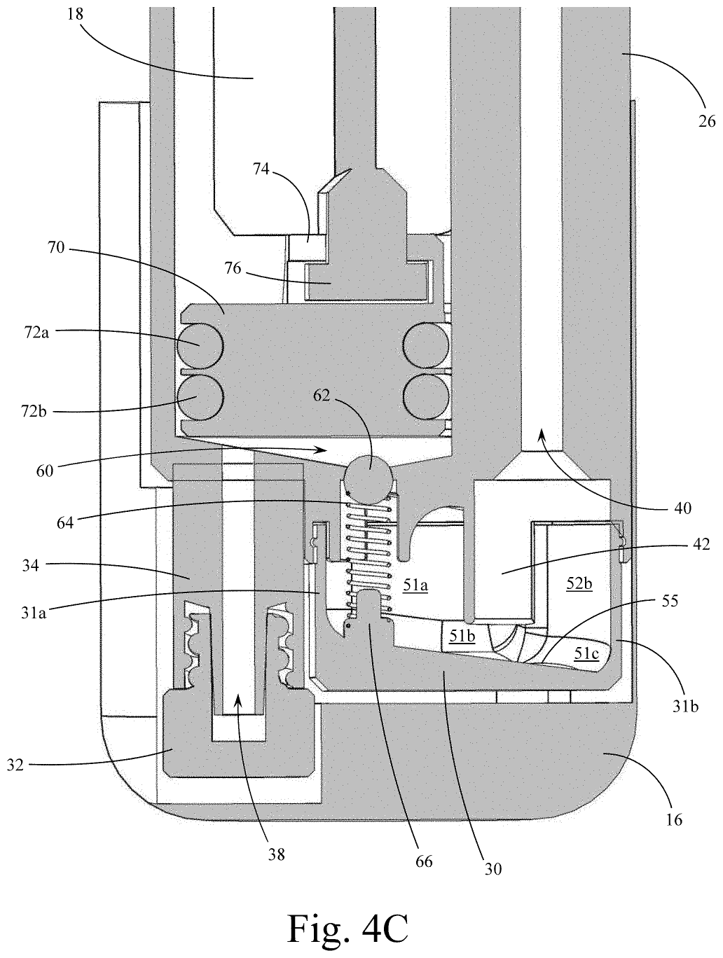

Riser tube 26 terminates with riser tube port 40 that opens into a chamber defined by the gutter cap (not shown in FIG. 2A) and curved wall baffle 42. Gutter curved wall slots 80a & 80b accommodate insertion of the walls of the gutter cap as shown in more detail in the cross section of FIG. 4C. Outside curved wall baffle 42 are intermediate gutter chambers 52a & 52b partially defined by curved gutter chamber walls 53a & 53b. Between these defined chamber spaces are structured valve spring keeper channel 48 and valve spring keeper wall 46, which (as seen best in FIG. 4C) help to position the ball valve components of the device while allowing the valve to open in an outflow direction from the large syringe. The valve opens and closes large tube outlet port (valved) 50 in the manner described in more detail below to transfer the centrifuged cell pellet material from the large syringe into the gutter chambers and channels (and eventually into the small transfer syringe).

FIG. 2B is a perspective view of the interior of the gutter cap of the first preferred embodiment of the dual syringe assembly of the present invention. Gutter cap 30 mates with and covers the lower portion of the dual syringe body shown open in FIG. 2A. Gutter cap 30 is configured with gutter square wall 31a and gutter curved wall 31b closed by gutter funnel floor 55 and gutter funnel walls 57. Positioned on gutter funnel floor 55 is valve spring post 66 which supports the valve spring that engages the valve ball described above to open and close large tube outlet port (valved) 50. Gutter cap 30 is preferably configured with ridged edges on the peripheral walls that snap inside matching interior grooves in the walls of the open enclosure positioned on the base of the dual syringe structure (seen best in FIG. 2A).

FIG. 2C is an end view of the first preferred embodiment of the dual syringe assembly of the present invention as shown in FIG. 1A with the end cover, luer stop, valve components, and gutter cap removed for clarity. In this view, the lower ends of large (harvesting) syringe tube 24 and riser tube 26 are shown in profile. As described above, large syringe tube 24 transitions into luer (cannula connector) 34 which is internally configured with luer port 38. Riser tube 26 terminates with riser tube port 40 that opens into a chamber defined by the gutter cap and curved wall baffle 42. Once again, gutter curved wall slots 80a & 80b are large enough to just accommodate insertion of the walls of the gutter cap to close off the chambers and channels. Outside curved wall baffle 42 are intermediate gutter chambers 52a & 52b partially defined by curved gutter chamber walls 53a & 53b. Between these defined chamber spaces are structured valve spring keeper channel 48 and valve spring keeper wall 46, which serve to position the ball valve components of the device while allowing the valve to open and close large tube outlet port (valved) 50 to transfer the centrifuged cell pellet material from the large syringe into the gutter chambers and channels.

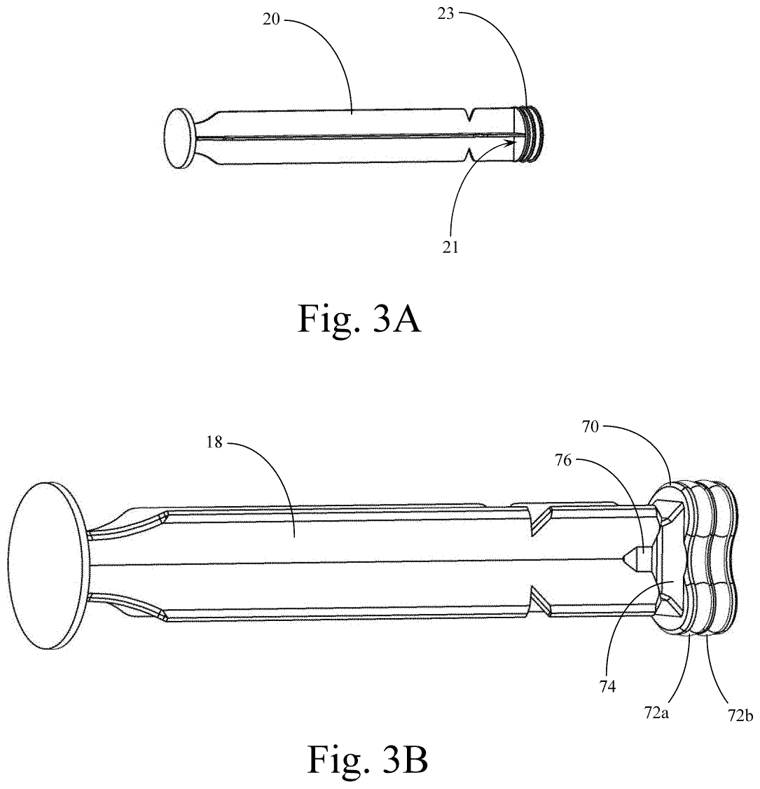

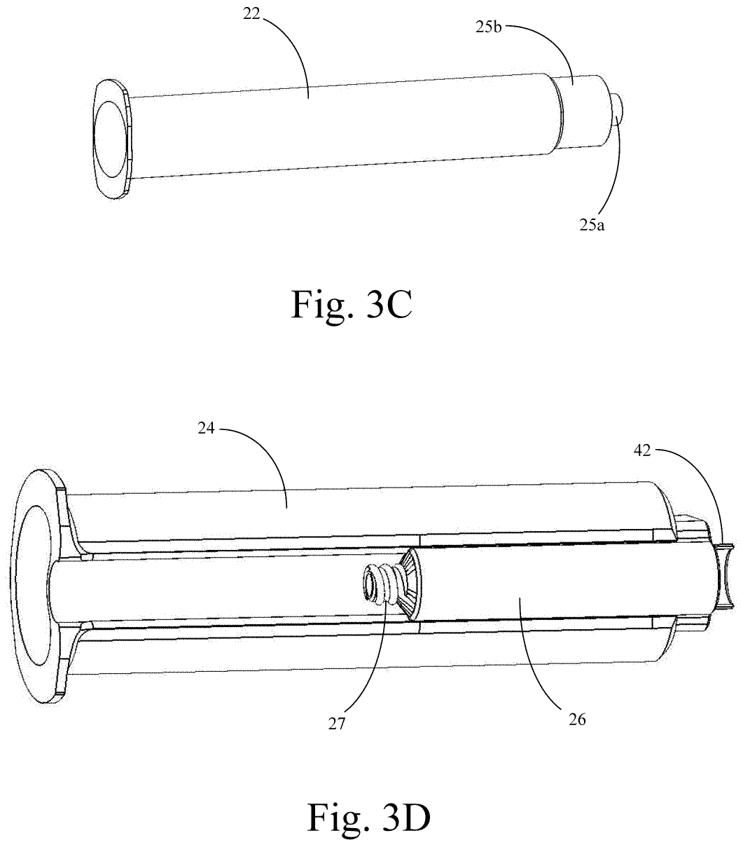

FIGS. 3A-3D are detailed perspective views of the syringe components of the first preferred embodiment of the dual syringe assembly of the present invention as shown in FIG. 1A. In FIG. 3A, small syringe shaft 20 is shown attached to small syringe plunger 23 with small syringe plunger attachment post 21 a manner typical of most cylindrical syringes. The combination of shaft 20 and plunger 23 are inserted into small syringe tube 22 shown in detail in FIG. 3C. Small syringe tube 22 is attached to riser tube by a threaded connection or other twist to lock syringe connectors structured in luer (riser tube connector) 25a and small syringe riser tube attachment collar 25b.

In FIG. 3B, large syringe shaft 18 is shown removably attached to large syringe plunger 70 which holds large syringe plunger gasket rings 72a & 72b. The separable attachment components made up of large syringe plunger attachment collar 74 and mating large syringe plunger attachment post 76 are preferably a slotted arrangement that allows the shaft to be slid to the slide (when the shaft is sufficiently drawn out from the syringe tube) and disconnected from the plunger. As described above, the shape of the large syringe plunger 70 accommodates the interior cross section of large syringe tube 24 (see FIG. 3D) into which the shaft and plunger are inserted. Attached to or integral with large (harvesting) syringe tube 24 is riser tube 26. As seen in FIG. 3D, the upper end of riser tube 26 is configured with small syringe riser tube connection threads 27 while the lower end transitions into curved wall baffle 42 as described above.

FIG. 4A is a cross sectional view of the first preferred embodiment of the dual syringe assembly of the present invention as shown in FIG. 1A with all components in place as they would be prior to use of the device. This view is a cross section taken generally along a plane of symmetry dividing each of the syringes with a centerline through the large tube outlet port of the large (harvesting) syringe. Dual syringe assembly 10 includes large (harvesting) syringe 12 and small (transfer) syringe 14. In this assembly prior to use the dual syringe is covered with end cover (sabot) 16. Large (harvesting) syringe 12 is generally made up of large (harvesting) syringe shaft 18 and large (harvesting) syringe tube 24. Small (transfer) syringe 14 is generally made up of small (transfer) syringe shaft 20 and small (transfer) syringe tube 22. Small syringe 14 is removably positioned in a channel formed along the length of large syringe 12. Riser tube 26 extends up from the common base to a point of attachment for small syringe 14. As can be seen in this view, the riser tube arrangement permits large syringe shaft 18 and small syringe shaft 20 to be positioned proximate to each other for handling, use and operation of the dual syringe.

In FIG. 4A, large syringe shaft 18 is shown removably attached to large syringe plunger 70 which holds large syringe plunger gasket rings. The separable attachment components made up of large syringe plunger attachment collar 74 and mating large syringe plunger attachment post 76 are shown connected. As described above, the shape of the large syringe plunger 70 accommodates the interior cross section of large syringe tube 24 into which the shaft and plunger are inserted. Because of this non-circular cross section of large syringe tube 24, a portion of the shaft 18 is hidden behind small syringe 14 and riser tube 26 in this view. Riser tube 26 is attached to or integral with large (harvesting) syringe tube 24. Riser tube port 40 is the aperture through which the cell pellet material enters the small transfer syringe 14.

The lower portion of large syringe tube 24 and riser tube 26 is structured to provide the flow chambers and channels (when closed off with gutter cap 30) that direct the flow of cell pellet material from the harvesting syringe (post centrifuging) into the transfer syringe. The tissue material will initially be present (post centrifuging) in the variable volume within the large syringe tube 24 between the plunger 70 and the floor of the tube around the ball valve. This large syringe variable volume 60 is minimized in the view of FIG. 4A and highlights the slope of the floor down to the ball valve. Also of note is the positioning of the inlet port into variable volume 60 that extends up from luer port 38. Not only is this inlet port offset from a centerline of the large syringe tube it is positioned higher on the floor of the tube than the outlet port fitted with the ball valve. These structures facilitate the flow of a maximum amount of cell pellet material out of the large harvesting syringe and through the transfer chambers and channels. With luer stop (cannula connector cover) 32 in place as shown in FIG. 4A, there is little or no outflow back though the inlet port. Also shown in FIG. 4A (in cross section) are the gutter cap 30 and the end cover 16.

FIG. 4B is a detailed cross sectional view of the small syringe-riser tube connection of the first preferred embodiment of the dual syringe assembly of the present invention. In this cross section view, small syringe shaft 20 is shown attached to small syringe plunger 23 with small syringe plunger attachment post 21 a manner typical of most cylindrical syringes. The combination of shaft 20 and plunger 23 are positioned in small syringe tube 22. Small syringe tube 22 is attached to riser tube by a threaded connection 27 (or other twist to lock syringe connector) structured in luer (riser tube connector) 25a and small syringe riser tube attachment collar 25b. The variable volume 61 within the small syringe tube 22 between the plunger 23 and the floor of the tube is minimized in the view of FIG. 4B as it would be prior to use of the device and prior to a transfer of the collected and centrifuged material. In FIG. 4B, the upper end of riser tube port 40 opens into small (transfer) syringe luer port 29. No valve is required at this connection as material flows in and out of the small syringe through this single port (in contrast to the large syringe which has a closeable inlet port and a valved outlet port.

FIG. 4C is a detailed cross sectional view of end portion of the first preferred embodiment of the dual syringe assembly of the present invention. In FIG. 4C, the lower end of large syringe shaft 18 is shown removably attached to large syringe plunger 70 which holds large syringe plunger gasket rings 72a & 72b. The separable attachment components made up of large syringe plunger attachment collar 74 and mating large syringe plunger attachment post 76 are shown connected. Riser tube 26 is attached to the side of or integral with large (harvesting) syringe tube 24. Riser tube port 40 is the aperture through which the cell pellet material enters the small transfer syringe 14 (not seen in this detailed view). The lower portion of large syringe tube 24 and riser tube 26 is closed off with the walls 31a & 31b of gutter cap 30 snapped into place in a manner that directs the flow of cell pellet material from the harvesting syringe into the transfer syringe.

The harvesting of tissue material into the large syringe occurs as described above through luer port 38 up through luer (cannula connector) 34 which opens into variable volume 60. In the view of FIG. 4C, and except while harvesting material, luer port 38 is covered and sealed with luer stop (cannula connector cover) 32. Once harvested and centrifuged, a flow of material out from variable volume 60 may be effected through the large syringe tube outlet port by way of the valve made up of valve ball 62 and valve spring 64 positioned on valve spring post 66. This flow passes into and through gutter flow chambers 51a-51c and then into intermediate gutter chambers 52a & 52b (only 52b shown in FIG. 4C). This flow is moderated by the presence of curved wall baffle 42 which directs the flow downward against gutter funnel floor 55 before moving upward and entering the transfer syringe by way of riser tube 26 through riser tube port 40. This moderated flow is important for the controlled transfer of only the cell pellet materials from the large harvesting syringe (post centrifuging) to the small transfer syringe. Unmoderated flow could result in the remixing of fluids and materials post centrifuging in a manner that defeats the purpose of the centrifuging process.

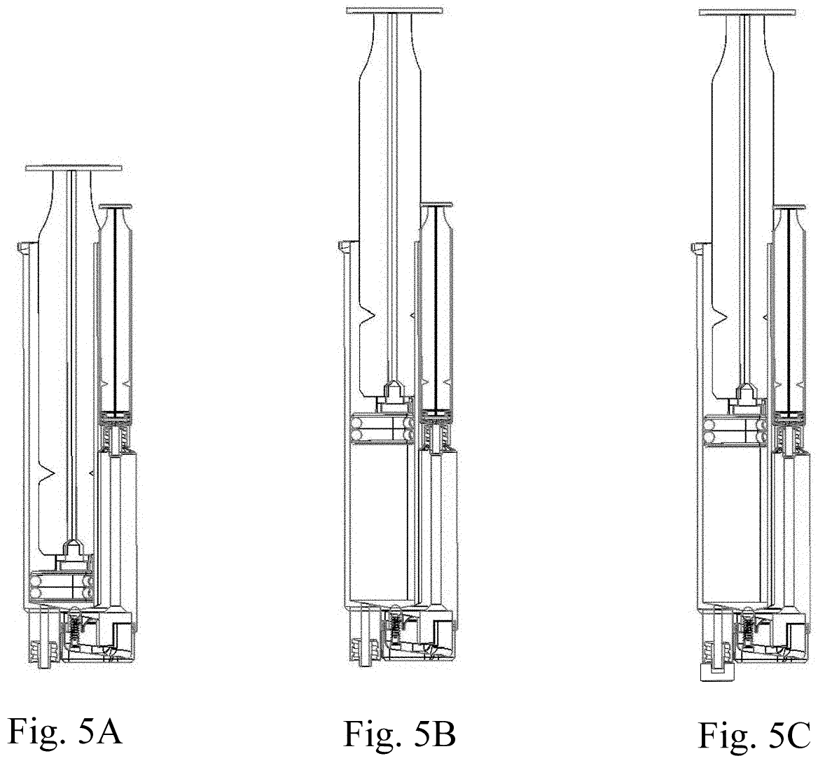

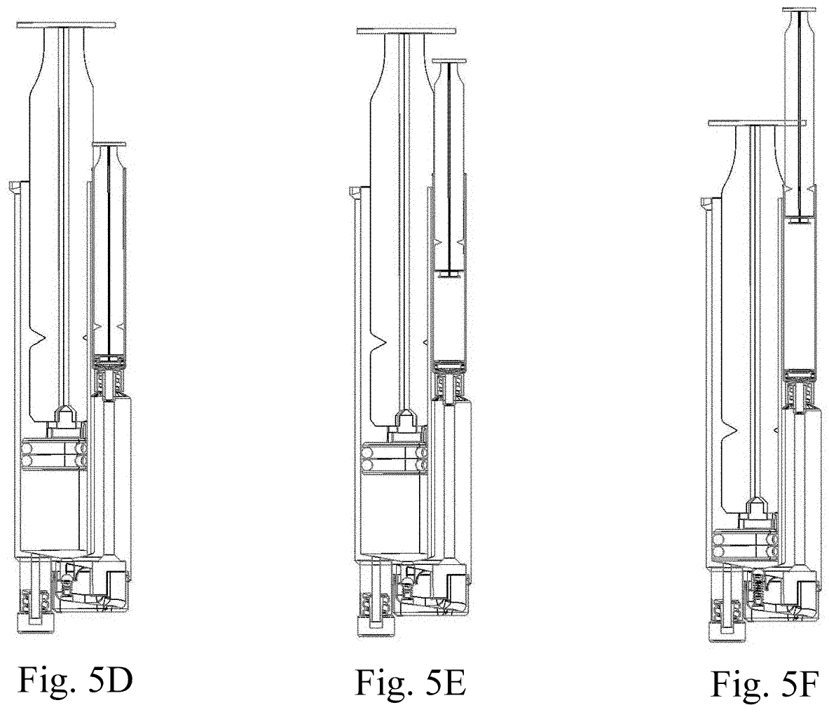

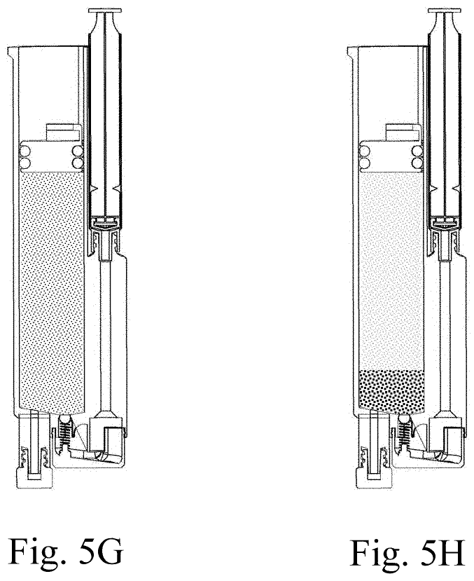

Reference is next made to FIGS. 5A-5H for a series of cross sectional views that track the motion and positioning of the components in the dual syringe system of the present invention as the process for harvesting, centrifuging, and transferring cell material is carried out. FIG. 5A is a cross section view of the device of the present invention configured to begin harvesting with the attachable cannula assembly removed for clarity. In this pre-harvesting condition, both the large and small syringes are compressed, minimizing the volume within each. Harvesting occurs as the large syringe shaft is drawn out and material from the patient is drawn into the interior volume of the large syringe through the offset inlet port.

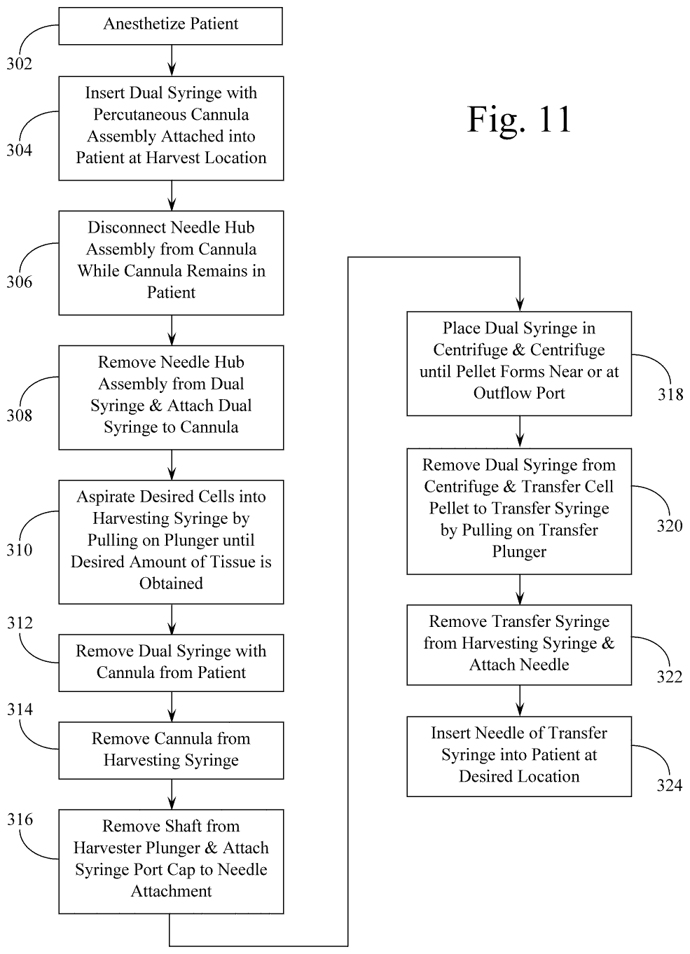

FIG. 5B is a cross section view of the device of the present invention configured post harvesting (and/or during harvesting) with the large syringe drawn out (once again the attachable cannula is removed for clarity). FIG. 5B generally aligns with Step 310 in FIG. 11 described below as the large syringe is slowly filled by the harvesting of cell material from one or more locations in the patient. FIG. 5C is a cross section view of the device of the present invention configured post harvesting with the large syringe drawn out and the luer stop positioned in place of the attachable cannula, the device prepared for centrifuging. FIG. 5C generally aligns with Step 316 in FIG. 11 described below.

Reference is next made specifically and separately to FIGS. 5G & 5H for a description of the motion and positioning of the elements in the dual syringe system of the present invention as the process for centrifuging cell material is carried out. FIG. 5G is a cross section view of the device of the present invention configured for centrifuging with the large syringe shaft removed before centrifuging begins. A relatively homogenous mixture of cell material and tissue fluids can be seen in the interior of the harvesting syringe. The harvesting syringe shaft is removed for ease of use in typical centrifuge instruments. FIG. 5H is a cross section view of the device of the present invention configured immediately after centrifuging with the large syringe shaft removed and the cell pellet material forced to the lower part of the large syringe. FIGS. 5G & 511 generally align with Step 318 in FIG. 11 described below.

Referring back to FIGS. 5D-5F, FIG. 5D is a cross section view of the device of the present invention configured post centrifuging with the small syringe starting to be drawn out and the large syringe (in response to the negative pressure and/or separately pushed) moved partially inward, opening the check valve and initiating a flow of the pellet material into the gutter chambers. FIG. 5E is a cross section view of the device of the present invention configured post centrifuging with the large syringe shaft and plunger moved partially inward, with the transfer flow moving through the riser tube into the small syringe. FIG. 5F is a cross section view of the device of the present invention configured post transfer from the large syringe with the large syringe shaft and plunger moved inward, the transfer flow moving through the riser tube to fill the small syringe. FIGS. 5D-5F generally align with Step 320 in FIG. 11 described below. The filled small syringe is then removed from the dual syringe assembly for use injecting the cell materials into the patient at the desired locations (again see Steps 322 & 324 in FIG. 11 described below).

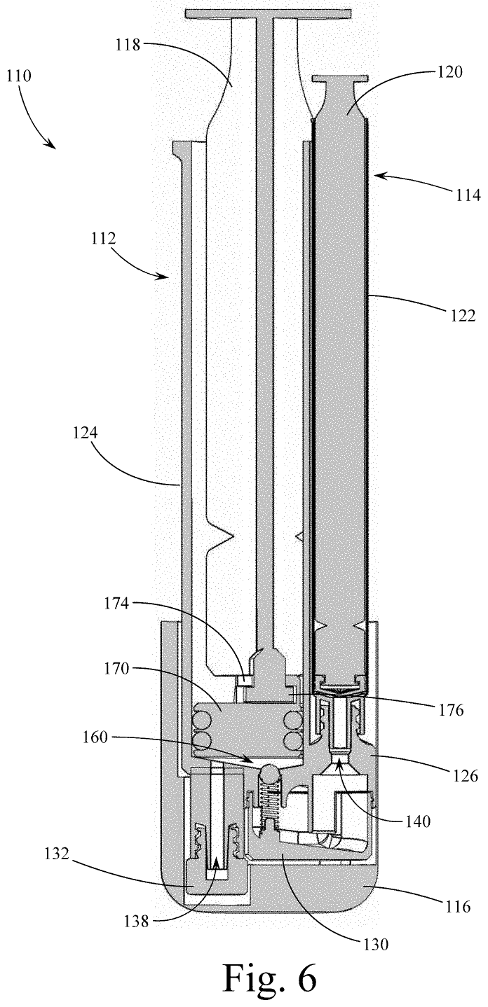

FIG. 6 is a cross sectional view of a second preferred embodiment of the dual syringe assembly of the present invention with a lower step riser tube and an elongated transfer syringe. In FIG. 6, large syringe shaft 118 is shown removably attached to large syringe plunger 170 which holds large syringe plunger gasket rings. The separable attachment components made up of large syringe plunger attachment collar 174 and mating large syringe plunger attachment post 176 are shown connected. As described above, the shape of the large syringe plunger 170 accommodates the interior cross section of large syringe tube 124 into which the shaft and plunger are inserted. Because of this non-circular cross section of large syringe tube 124, a portion of the shaft 118 is hidden behind small syringe 114 in this view. Riser tube 126 is short in this embodiment and is preferably integral with large (harvesting) syringe tube 124. Riser tube port 140 is the aperture through which the cell pellet material enters the small transfer syringe 114.

The lower portion of large syringe tube 124 and the shorter riser tube 126 is structured to provide the flow chambers and channels (when closed off with gutter cap 130) that direct the flow of cell pellet material from the harvesting syringe (post centrifuging) into the transfer syringe. The tissue material will initially be present (post centrifuging) in the variable volume within the large syringe tube 124 between the plunger 170 and the floor of the tube around the ball valve. This large syringe variable volume 160 is minimized in the view of FIG. 6 and highlights the slope of the floor down to the ball valve. Also of note is the positioning of the inlet port into variable volume 160 that extends up from luer port 138. Not only is this inlet port offset from a centerline of the large syringe tube it is positioned higher on the floor of the tube than the outlet port fitted with the ball valve. These structures facilitate the flow of a maximum amount of cell pellet material out of the large harvesting syringe and through the transfer chambers and channels. With luer stop (cannula connector cover) 132 in place as shown in FIG. 6, there is little or no outflow back though the inlet port. Also shown in FIG. 6 (in cross section) are the gutter cap 130 and the end cover 116.

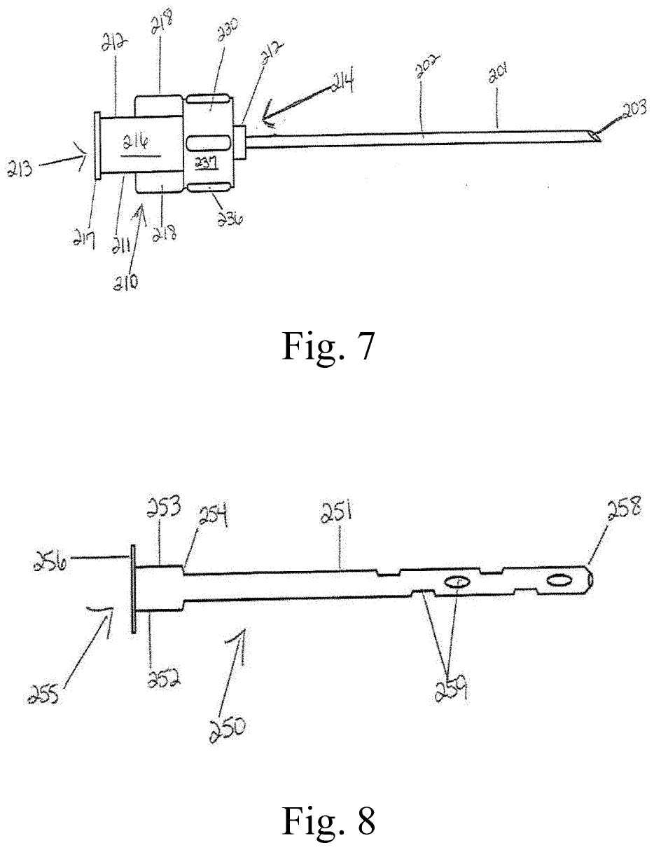

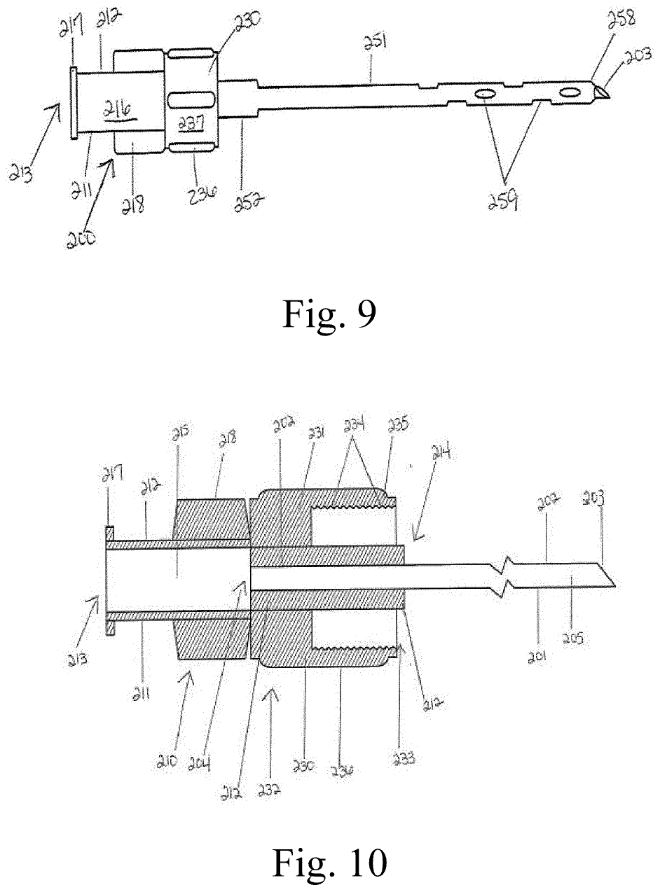

As seen in FIG. 9, the percutaneous cannula assembly 200 is composed of a needle hub 210, a needle 201, and a removable cannula 250. As seen in FIGS. 7 & 10, the hollow tubular needle 201 is comprised of cylindrical sidewall 202 having a beveled point 203 on one end and an opening 204 on the opposing end. A cavity 205 is formed by the cylindrical sidewall 202 and extends from the beveled point 203 to the opening 204. As seen in FIGS. 7, 9 &10, the needle hub 210 contains a syringe connecting portion 211 and a cannula connecting portion 230. The syringe connecting portion 211 consists of a cylindrical sidewall 212 having a connecting outlet 213 on one end and a needle receiving outlet 214 on the opposing end. A radial flange 217 is positioned around the connecting outlet 213 on the exterior face 216 of the cylindrical sidewall 212. The radial flange 217 may be consistent with a female luer taper connection. A cavity 215 is formed by the cylindrical sidewall 212 and extends from the connecting outlet 213 on one end and a needle receiving outlet 214. The needle 201 is permanently affixed to at least a portion of the interior face of the cylindrical sidewall 212. Two wings 218 extend radially away from the exterior face 216 of the cylindrical sidewall 212. The wings 218 permit a user to grip and rotate the needle hub 210. The wings 218 are positioned on the exterior face 216 of the cylindrical sidewall 212 at a sufficient distance away from the radial flange 217 to allow the radial flange 217 to connect to the needle attachment on the dual syringe and be screwed or positioned into place. A fluid communication pathway exists from the connecting outlet 213 to the beveled point 203 of the needle 201.

As seen in FIG. 8, the cannula connecting portion 230 consists of a cylindrical sidewall 231 having a connecting end 232 on one end and a cannula receiving end 233 on the opposing end. The diameter of the cylindrical sidewall 231 of the cannula connecting portion 230 is greater than the cylindrical sidewall 212 of the syringe connecting portion 211. The connecting end 232 is attached to the exterior face 216 of the cylindrical sidewall 212 of the syringe connecting portion 211 at the approximate midpoint of the cylindrical sidewall 212 of the syringe connecting portion 211. Threads 234 are positioned on the interior face 234 of the cylindrical sidewall 231 of the cannula connecting portion 230. Threads 234 may be consistent with a male luer lock taper connection. Radial ridges 236 are positioned on the external face 237 of the cylindrical sidewall 231 of the cannula connecting portion 230. The radial ridges 236 assist in gripping the needle hub 210. The distance between the interior face 234 of the cylindrical sidewall 231 of the cannula connecting portion 230 and the exterior face 216 of the cylindrical sidewall 212 of the syringe connecting portion 211 is of sufficient distance that a cannula or other syringe may be removably attached to the needle hub 210.

As seen in FIG. 8, the cannula 250 is comprised of a tubular shaft 251 attached to a cannula hub 252. The cannula hub 252 is tubular in shape with a generally cylindrical sidewall 253 with a cannula receiving portion 254 on one end and outlet 255 on the opposing end. A radial flange 256 is positioned around the outlet 255 of the cylindrical sidewall 253. The radial flange 256 may be consistent with a female luer taper connection. One end of the tubular shaft 251 is attached to the cannula hub 252 at the cannula receiving portion 254. The other end of the tubular shaft 251 is a tapered outlet 258. The cylindrical sidewall 253 and cannula shaft 251 form a cavity 257 between the outlet 255 and the tapered outlet 258. The cannula shaft 251 and the tapered outlet 258 have an inner diameter large enough to allow the needle 201 to pass through. Ports 259 extend longitudinally through the sidewall of the tubular shaft 251. Ports 259 may have smooth edges for minimal tissue cleavage or sharp skived edges for maximum tissue recovery depending on the desired use and desired tissue and/or cells to harvest. Ports 259 may be oriented on one side or may extend generally around the circumference of the tubular shaft 251. Depth markings 260 are positioned along the outer face of the cannula shaft 251 that indicate the distance from the respective marking to the tapered outlet 258.

The needle hub 210 may attach to the dual syringe of the present invention through mating between the needle attachment of the dual syringe and ridge 217 of the needle hub assembly 210. The cannula 250 may attach to the needle hub 210 through mating of the cannula connecting portion 230 and radial flange 256 of the cannula 250. As seen in FIG. 9, the needle 201 extends out the tapered outlet 258 of the cannula 250 when the cannula 250 is attached to the needle hub 210. The cannula 250 may also attach to the dual syringe of the present invention through mating between the needle attachment of the dual syringe and radial flange 256 of the cannula 250. In the preferred embodiment, the attachments are luer lock taper connections although alternative embodiments may use a luer slip taper connection.

Referring to FIG. 11, the broad process of operation of the dual syringe comprises the following steps:

Anesthetize the patient (Step 302) generally or locally where cells are to be harvested. Anesthetization may occur through use of the dual syringe with a standard hypodermic needle attached, or through the percutaneous adipose aspirating cannula of the present invention. Anesthetizing agent is drawn into the harvesting syringe by pulling on the harvesting plunger. The anesthetic agent is then applied locally at the site of cell harvesting.

The dual syringe of the present invention, with the attached transfer syringe (with the transfer plunger fully depressed) and the percutaneous cannula assembly, is inserted (Step 304) into tissue or bone of a patient to harvest the desired cells. The beveled point of the needle along with the cannula is inserted percutaneously through the patient's skin and advanced into the patient until the appropriate depth as indicated by the depth markings on the cannula. The tapered outlet of the cannula assists insertion into the patient's skin to reduce any catching or blunt trauma.

Once the desired depth is obtained, the cannula is unmated (Step 306) from the needle hub assembly which allows for the removal of the dual syringe (with the needle hub assembly still attached) from the cannula. The needle hub assembly is unmated (Step 308) from the harvesting syringe. The harvesting syringe is then attached to the cannula through mating the needle attachment of the dual syringe and the cannula. This establishes a fluid communication pathway from the tapered outlet and ports, through the tubular shaft, through the cannula hub, through the outlet, through the inflow port, and into the barrel of the harvesting syringe.

The cannula may then be used as a standard aspirating cannula to harvest the desired cells (Step 310). The user creates negative pressure within the barrel by pulling on the harvesting plunger to draw in cells and tissues while simultaneously moving the cannula within the patient's body at the desired location. As the cannula is moved back and forth while inside the patient, the ports and tapered outlet shear and collect cells/tissue which are then drawn into the cannula and are ultimately drawn into the barrel.

Once the desired volume of cells and/or tissues are harvested, the harvesting syringe (with the cannula still attached) is removed (Step 312) from the patient.

The cannula is removed (Step 314) from the harvesting syringe. The shaft is separated from the plunger head and removed (Step 316) from the harvesting syringe. A standard syringe port cap is attached to the needle attachment to seal the inflow port. The cap ensures sterility and prevents migration of the cell pellet into the inflow port.

The dual syringe is placed in a centrifuge (Step 318) and spun at approximately 500 to 2000 g forces for a period of 3 to 20 minutes. Centrifugation causes the aspirated cells to separate from the aspirated fluid. As a result of the funnel shape of the bottom wall, the cells, denser than the fluid, form a pellet on the bottom wall near the outflow port. The dual syringe is removed (Step 320) from the centrifuge. The transfer plunger is pulled away from the bottom end which creates negative pressure within the interior cavity of the transfer syringe. This negative pressure opens the valve to allow cells of the pellet to move through the outflow port, through the pathways described above, into the interior cavity of the transfer syringe. To adjust for the varying hydraulic pressure within the interior cavity of the harvesting syringe, if necessary, the harvesting plunger may be manually depressed, after reattachment of the shaft, by the user in conjunction with user's pulling of the transfer plunger.

Once the cell pellet is fully transferred to the transfer syringe, the transfer syringe is removed (Step 322) from the syringe locking mechanism and a standard hypodermic needle is attached to the transfer syringe at the needle attachment. The gauge and design of the needle for the transfer syringe may depend on the type of tissue delivered, the tissue the needle needs to penetrate to deliver the cells and the volume of the cells to be delivered. Saline and/or other chemicals may be added to reconstitute the cell pellet in the transfer syringe.

The transfer syringe is then inserted (Step 324) into the patient in a specific location for delivery of the harvested cells for therapeutic purposes.

A person of ordinary skill in the art would appreciate the number, gauge, and design of the various ports may vary depending on the type of tissue harvested, the tissue the needle needs to penetrate to acquire the tissue, and the volume of the tissue needed.

The dual syringe may also be used with a standard aspirating cannula as well with traditional access to the cells created through trocar use or surgical incision. Once the cells are harvested, the dual syringe is removed from the patient. The standard aspirating cannula is removed from the harvesting syringe. The shaft is disconnected from the plunger head and removed from the harvesting syringe. A standard syringe port cap is attached to the needle attachment to seal inflow port. The dual syringe is placed in a centrifuge and spun at approximately 500 to 2000 g forces for a period of 3 to 20 minutes. Centrifugation causes the aspirated cells to separate from the aspirated fluid. As a result of the funnel shape of the bottom wall, the cells, denser than the fluid, form a pellet on the bottom wall near the outflow port. The dual syringe is removed from the centrifuge. The transfer plunger is pulled away from the bottom end which creates negative pressure within the interior cavity of the transfer syringe. This negative pressure opens the valve to allow cells of the pellet to move through the outflow port, through the pathways described above into the interior cavity of the transfer syringe. To adjust for the varying hydraulic pressure within the interior cavity of the harvesting syringe, if necessary, the harvesting plunger may be manually depressed, after reattachment of the shaft, by the user in conjunction with user pulling the transfer plunger. Once the cell pellet is fully transferred to the transfer syringe, the transfer syringe is removed from the syringe locking mechanism and a standard hypodermic needle is attached to the transfer syringe at the needle attachment. The transfer syringe is then inserted into a patient in a specific location for delivery of the harvested cells for therapeutic purposes.

The above device may be used for autologous stem cell transplantation in an office setting according to the method generally described in FIG. 11. A healthcare provider may utilize local anesthetic at the specific harvesting site or general anesthetic for the harvesting of stem cells. Stem cell harvesting may occur through adipose tissue in a patient's abdominal region, or through adipose tissue in another adipose-rich region such as the inner thigh or through a patient's bone marrow. The healthcare provider utilizes the sterile dual syringe and attaches the percutaneous cannula of the present invention. The healthcare provider inserts the dual syringe with the percutaneous cannula as described in FIG. 11 into the patient's abdomen and aspirates 50 to 60 milliliters of adipose tissue into the harvesting syringe. Centrifugation separates a cellular pellet rich in mesenchymal stem cells from the stromal vascular fraction. The mesenchymal stem cells are then transferred to the transfer syringe as described in FIG. 11. The transfer syringe is removed from the harvesting syringe, a needle is attached, and the mesenchymal stem cells are transplanted into the same patient for treatment of osteoarthritis, for assistance in wound healing, or for one or more of many other regenerative medicine uses. The procedure is accomplished in the same office visit for the patient.

In a preferred embodiment the dual syringe assembly (harvesting syringe and transfer syringe), percutaneous cannula assembly, syringe port cap, and hypodermic needle are sterile and stored in a single use sterile packaging kit. The packaged unit is designed for singular use for a single patient for transplantation of autologous tissue.

The interior surfaces of the sidewalls of the syringe tubes are generally smooth and correspond in shape to the exterior surfaces associated with each syringe. A ridge may be located along the interior surface of the cylindrical sidewalls near the top end. The cylindrical sidewalls are generally translucent and contain identifying markings to indicate the volume of material within the interior cavity. The riser tube may be bonded, glued, or welded onto the large syringe tube or may be integrally molded with the tube. At least a portion of the longitudinal cylindrical sidewall of the tube of the transfer syringe fits within the hemicylindrical groove of the harvesting syringe.

As can be seen from the foregoing, the aspects, concepts, features, and elements of the present invention may be embodied in a variety of structures, a variety of arrangements, and its methods done in a variety of ways. It involves structures, systems, method steps, and techniques to accomplish the appropriate ends and desired goals. Techniques and method steps according to the present invention are disclosed as part of the results to be achieved by the various structures and described and as steps which for utilization of the structures as intended and described. In addition, while some structures are disclosed, it should be understood that these not only accomplish certain methods, but also can be varied in a number of ways within the scope of the present invention. It should also be understood that a variety of changes may be made without departing from the scope of the invention. Such changes are also implicitly included in the description and still fall within the spirit and scope of this invention.

I claim:

1. A dual syringe assembly comprising: a first syringe having an open top end, a longitudinal sidewall, a bottom end opposite said open top end wherein said sidewall of said first syringe defines a cavity within said first syringe; a first port positioned at said bottom end and a second port positioned at said bottom end below said first port; a plunger inserted into said cavity of said first syringe through said open top end; a second syringe having an open top end, a longitudinal sidewall, a bottom end opposite said open top end wherein said sidewall of said second syringe defines a cavity within said second syringe; a port positioned at said bottom end of said second syringe; a plunger inserted into said cavity of said second syringe through said top open end; said first port of said first syringe creating a fluid pathway between said cavity of said first syringe and a needle hub attached to said first syringe; and said second port of said first syringe creating a fluid pathway between said cavity of said first syringe and said port of said second syringe.

2. The dual syringe assembly of claim 1: wherein said longitudinal sidewall of said second syringe is cylindrical; and wherein at least a portion of said longitudinal sidewall of said second syringe is removably positioned within a longitudinal groove on the exterior surface of said longitudinal sidewall of said first syringe.

3. The dual syringe assembly of claim 1: wherein said first port and said second port of said first syringe extend through a bottom wall positioned at said bottom end of said first syringe; and wherein said bottom wall is positioned opposite said open top end of said first syringe.

4. The dual syringe assembly of claim 3: wherein said bottom wall of said first syringe has an interior face that slopes away from said open top end from said first port to said second port.

5. The dual syringe assembly of claim 1: wherein said plunger of said first syringe comprises a shaft with a plunger head on one end and a handle on the opposing end; and wherein said shaft is removable from said plunger head.

6. The dual syringe assembly of claim 1: wherein said needle hub further comprises a syringe attachment member; a cannula attachment member; and a hollow needle extending from said cannula attachment member and creating a fluid pathway between said first port of said first syringe and said needle.

7. The dual syringe assembly of claim 6 further comprising: a cannula having an opening on one end and an attachment member on the opposing end; wherein said attachment member of said cannula is connected to said cannula attachment member and said needle is positioned within said cannula and extends through said opening of said cannula; and wherein said syringe attachment member is attachable to and detachable from said first syringe.

8. A dual syringe assembly of claim 7 wherein said cannula is removable from said needle hub.

9. A dual syringe assembly of claim 7 wherein said cannula further comprises at least one port along the longitudinal axis.

10. A dual syringe assembly of claim 7 wherein said cannula tapers to said opening of said cannula.

11. A dual syringe assembly of claim 7 wherein said cannula is attachable to and detachable from said first syringe.

12. A dual syringe assembly of claim 1 further comprising: a cannula having an opening on one end and an attachment member on the opposing end wherein said attachment member of said cannula is attachable to and detachable from said first syringe; and a fluid pathway is between said cannula and said first port of said first syringe.

13. A dual syringe assembly of claim 12 wherein said cannula further comprises at least one port along the longitudinal axis.

14. A dual syringe assembly of claim 12 wherein said cannula tapers to said opening of said cannula.

15. A dual syringe assembly comprising: a first syringe having an open top end, a longitudinal sidewall, and a bottom wall opposite said open top end wherein said sidewall and said bottom wall define a cavity within said first syringe; a first port and a second port extending through said bottom wall of said first syringe; a plunger inserted into said cavity of said first syringe through said open end; a second syringe having an open top end, a longitudinal sidewall, and a bottom end opposite said open top end wherein said longitudinal sidewall defines a cavity within said second syringe; a port at said bottom end of said second syringe; a plunger inserted into said cavity of said second syringe through said open end; said first port of said first syringe creating a fluid pathway between said cavity of said first syringe and a needle attachment; and said second port of said first syringe creating a fluid pathway between said cavity of said first syringe and said port of said second syringe.

16. The dual syringe assembly of claim 15 wherein said second port of said first syringe is positioned below said first port of said first syringe.

17. The dual syringe assembly of claim 15 wherein said longitudinal sidewall of said second syringe is cylindrical and at least a portion of which is removably positioned within a longitudinal groove on the exterior surface of said longitudinal sidewall of said first syringe.

18. The dual syringe assembly of claim 15 wherein said bottom wall of said first syringe has an interior face that slopes away from said open top end from said first port to said second port.

19. A dual syringe assembly compromising: a first syringe having an open top end, a bottom end opposite said open top end and a longitudinal sidewall wherein said longitudinal sidewall defines a cavity within said syringe; a plunger inserted into said cavity of said first syringe through said open end; a first port at said bottom end of said first syringe creating a fluid pathway into said cavity of said first syringe; a second port at said bottom of end of said first syringe creating a fluid pathway between said cavity of said first syringe and a cavity of a second syringe; said second syringe having an open top end, a bottom end opposite said open top end and a longitudinal cylindrical sidewall wherein said cylindrical sidewall defines a cavity within said second syringe; a plunger inserted into said cavity of said second syringe through said open top end; a port at said bottom end of said second syringe creating said fluid pathway between said cavity of said second syringe and said cavity of said first syringe; and a longitudinal groove in the exterior surface of said sidewall of said first syringe wherein at least a portion of said cylindrical sidewall of second syringe is positioned within said groove.

20. The dual syringe assembly of claim 19 wherein said first port is positioned above second port.
